(19) United States
(12) Patent Application Publication
(10) Pub. No.: US 2008/0065083 A1
(43) Pub. Date: Mar. 13, 2008

(54) BONE TREATMENT SYSTEMS AND METHODS

(76) Inventors: Csaba Truckai, Saratoga, CA (US); Robert Luzzi, Pleasanton, CA (US); John H. Shadduck, Tiburon, CA (US)

Correspondence Address: KNOBBE MARTENS OLSON & BEAR LLP, 2040 MAIN STREET, FOURTEENTH FLOOR, IRVINE, CA, 92614, US

(21) Appl. No.: 11/852,067

(22) Filed: Sep. 7, 2007

Related U.S. Application Data
(60) Provisional application No. 60/842,804, filed on Sep. 7, 2006.
(60) Provisional application No. 60/842,805, filed on Sep. 7, 2006.
(60) Provisional application No. 60/899,763, filed on Feb. 6, 2007.

(51) Int. Cl. A61B 17/56; A61B 17/58; A61M 31/00

(52) U.S. Cl. 606/86; 604/513; 606/79; 606/93; 606/94

(57) ABSTRACT

The present invention relates in certain embodiments to systems for treating vertebral compression fractures. In one embodiment, an elongated sleeve defines a passageway therethrough, and has a threaded portion configured to engage bone. The sleeve includes a seal configured to allow instrument exchange through the passageway and into the interior of the vertebra to perform at least one medical procedure, such as injection of bone cement into the vertebral body.

### CROSS-REFERENCE TO RELATED APPLICATIONS

This application claims the benefit of U.S. Provisional Patent Application No. 60/842,804 filed Sep. 7, 2006, U.S. Provisional Application No. 60/842,805, filed Sep. 7, 2006, and U.S. Provisional Application No. 60/899,763 filed Feb. 6, 2007, the entire contents of which are incorporated herein by reference and should be considered a part of this specification. This application is related to the following U.S. patent applications: application Ser. No. 11/165,651 filed Jun. 24, 2005; application Ser. No. 11/165,652 filed Jun. 24, 2005; application Ser. No. 11/208,448 filed Aug. 20, 2005; application Ser. No. 60/713,521 filed Sep. 1, 2005; application Ser. No. 11/209,035 filed Aug. 22, 2005; and application Ser. No. 11/469,764 filed Sep. 1, 2006. The entire contents of all of the above applications are hereby incorporated by reference and should be considered a part of this specification.

### BACKGROUND OF THE INVENTION

1. Field of the Invention

The present invention relates to systems and methods for treating bone and more particularly to systems and methods for treating vertebral compression fractures. In one embodiment, an elongated sleeve provides a port that can be threadably engaged with cortical bone of a pedicle to allow instrument exchange through the port into the interior of the vertebra.

2. Description of the Related Art

Osteoporotic fractures are prevalent in the elderly, with an annual estimate of 1.5 million fractures in the United States alone. These include 750,000 vertebral compression fractures (VCFs) and 250,000 hip fractures. The annual cost of osteoporotic fractures in the United States has been estimated at $13.8 billion. The prevalence of VCFs in women age 50 and older has been estimated at 26%. The prevalence increases with age, reaching 40% among 80-year-old women. Medical advances aimed at slowing or arresting bone loss from aging have not provided solutions to this problem. Further, the population affected will grow steadily as life expectancy increases. Osteoporosis affects the entire skeleton but most commonly causes fractures in the spine and hip. Spinal or vertebral fractures also cause other serious side effects, with patients suffering from loss of height, deformity and persistent pain which can significantly impair mobility and quality of life. Fracture pain usually lasts 4 to 6 weeks, with intense pain at the fracture site. Chronic pain often occurs when one vertebral level is greatly collapsed or multiple levels are collapsed.

Postmenopausal women are predisposed to fractures, such as in the vertebrae, due to a decrease in bone mineral density that accompanies postmenopausal osteoporosis. Osteoporosis is a pathologic state that literally means “porous bones”. Skeletal bones are made up of a thick cortical shell and a strong inner meshwork, or cancellous bone, of with collagen, calcium salts and other minerals. Cancellous bone is similar to a honeycomb, with blood vessels and bone marrow in the spaces. Osteoporosis describes a condition of decreased bone mass that leads to fragile bones which are at an increased risk for fractures. In an osteoporosis bone, the sponge-like cancellous bone has pores or voids that increase in dimension making the bone very fragile. In young, healthy bone tissue, bone breakdown occurs continually as the result of osteoclast activity, but the breakdown is balanced by new bone formation by osteoblasts. In an elderly patient, bone resorption can surpass bone formation thus resulting in deterioration of bone density. Osteoporosis occurs largely without symptoms until a fracture occurs.

Vertebroplasty and kyphoplasty are recently developed techniques for treating vertebral compression fractures. Percutaneous vertebroplasty was first reported by a French group in 1987 for the treatment of painful hemangiomas. In the 1990's, percutaneous vertebroplasty was extended to indications including osteoporotic vertebral compression fractures, traumatic compression fractures, and painful vertebral metastasis. Vertebroplasty is the percutaneous injection of PMMA (polymethylmethacrylate) into a fractured vertebral body via a trocar and cannula. The targeted vertebrae are identified under fluoroscopy. A needle is introduced into the vertebrae body under fluoroscopic control, to allow direct visualization. A bilateral transpedicular (through the pedicle of the vertebrae) approach is typical but the procedure can be done unilaterally. The bilateral transpedicular approach allows for more uniform PMMA infill of the vertebra.

In a bilateral approach, approximately 1 to 4 ml of PMMA is used on each side of the vertebra. Since the PMMA needs to be is forced into the cancellous bone, the techniques require high pressures and fairly low viscosity cement. Since the cortical bone of the targeted vertebra may have a recent fracture, there is the potential of PMMA leakage. The PMMA cement contains radiopaque materials so that when injected under live fluoroscopy, cement localization and leakage can be observed. The visualization of PMMA injection and extravasation are critical to the technique—and the physician terminates PMMA injection when leakage is evident. The cement is injected using syringes to allow the physician manual control of injection pressure.

Kyphoplasty is a modification of percutaneous vertebroplasty. Kyphoplasty involves a preliminary step consisting of the percutaneous placement of an inflatable balloon tamp in the vertebral body. Inflation of the balloon creates a cavity in the bone prior to cement injection. The proponents of percutaneous kyphoplasty have suggested that high pressure balloon-tamp inflation can at least partially restore vertebral body height. In kyphoplasty, some physicians state that PMMA can be injected at a lower pressure into the collapsed vertebra since a cavity exists, when compared to conventional vertebroplasty.

The principal indications for any form of vertebroplasty are osteoporotic vertebral collapse with debilitating pain. Radiography and computed tomography must be performed in the days preceding treatment to determine the extent of vertebral collapse, the presence of epidural or foraminal stenosis caused by bone fragment retropulsion, the presence of cortical destruction or fracture and the visibility and degree of involvement of the pedicles.

Leakage of PMMA during vertebroplasty can result in very serious complications including compression of adjacent structures that necessitate emergency decompressive surgery. See “Anatomical and Pathological Considerations in Percutaneous Vertebroplasty and Kyphoplasty: A Reappraisal of the Vertebral Venous System”, Groen, R. et al, Spine Vol. 29, No. 13, pp 1465-1471 2004. Leakage or extravasion of PMMA is a critical issue and can be divided into paravertebral leakage, venous infiltration, epidural leakage and intradiscal leakage. The exothermic reaction of PMMA carries potential catastrophic consequences if thermal damage were to extend to the dural sac, cord, and nerve roots. Surgical evacuation of leaked cement in the spinal canal has been reported. It has been found that leakage of PMMA is related to various clinical factors such as the vertebral compression pattern, and the extent of the cortical fracture, bone mineral density, the interval from injury to operation, the amount of PMMA injected and the location of the injector tip. In one recent study, close to 50% of vertebroplasty cases resulted in leakage of PMMA from the vertebral bodies. See Hyun-Woo Do et al, “The Analysis of Polymethylmethacrylate Leakage after Vertebroplasty for Vertebral Body Compression Fractures”, Jour. of Korean Neurosurg. Soc. Vol. 35, No. 5 (May 2004) pp. 478-82, (http://www.jkns.or.kr/htm/abstract.asp?no=0042004086).

Another recent study was directed to the incidence of new VCFs adjacent to the vertebral bodies that were initially treated. Vertebroplasty patients often return with new pain caused by a new vertebral body fracture. Leakage of cement into an adjacent disc space during vertebroplasty increases the risk of a new fracture of adjacent vertebral bodies. See Am. J. Neuroradiol. February 2004; 25(2):175-80. The study found that 58% of vertebral bodies adjacent to a disc with cement leakage fractured during the follow-up period compared with 12% of vertebral bodies adjacent to a disc without cement leakage.

Another life-threatening complication of vertebroplasty is pulmonary embolism. See Bernhard, J. et al, “Asymptomatic diffuse pulmonary embolism caused by acrylic cement: an unusual complication of percutaneous vertebroplasty”, Ann. Rheum. Dis. 2003;62:85-86. The vapors from PMMA preparation and injection also are cause for concern. See Kirby, B, et al., “Acute bronchospasm due to exposure to polymethylmethacrylate vapors during percutaneous vertebroplasty”, Am. J. Roentgenol. 2003; 180:543-544.

In both higher pressure cement injection (vertebroplasty) and balloon-tamped cementing procedures (kyphoplasty), the methods do not provide for well controlled augmentation of vertebral body height. The direct injection of bone cement simply follows the path of least resistance within the fractured bone. The expansion of a balloon applies also compacting forces along lines of least resistance in the collapsed cancellous bone. Thus, the reduction of a vertebral compression fracture is not optimized or controlled in high pressure balloons as forces of balloon expansion occur in multiple directions.

In a kyphoplasty procedure, the physician often uses very high pressures (e.g., up to 200 or 300 psi) to inflate the balloon which crushes and compacts cancellous bone. Expansion of the balloon under high pressures close to cortical bone can fracture the cortical bone, typically the endplates, which can cause regional damage to the cortical bone with the risk of cortical bone necrosis. Such cortical bone damage is highly undesirable as the endplate and adjacent structures provide nutrients for the disc.

Kyphoplasty also does not provide a distraction mechanism capable of 100% vertebral height restoration. Further, the kyphoplasty balloons under very high pressure typically apply forces to vertebral endplates within a central region of the cortical bone that may be weak, rather than distributing forces over the endplate.

There is a general need to provide bone cements and methods for use in treatment of vertebral compression fractures that provide a greater degree of control over introduction of cement and that provide better outcomes. The present invention meets this need and provides several other advantages in a novel and nonobvious manner.

### SUMMARY OF THE INVENTION

Certain embodiments of the invention provide vertebroplasty systems and methods for sensing retrograde bone cement flows that can migrate along a fractured path toward a pedicle and risk leakage into the spinal canal. The physician can be alerted instantaneously of cement migration in a direction that can impinge on nerves or the spinal cord. Other embodiments include integrated sensing systems and energy delivery systems for applying energy to tissue and/or to bone cement that migrates in a retrograde direction wherein the energy polymerizes the cement and/or coagulates tissue to create a dam to prevent further cement migration. In another embodiment, the systems provide a cooling system for cooling bone cement in a remote container or injection cannula for controlling and extending the working time of a bone cement. In another embodiment, the bone cement injection system includes a thermal energy emitter for warming a chilled bone cement in the distal end of an injector or for applying sufficient energy to accelerate polymerization and thereby increase the viscosity of the bone cement.

In certain embodiments, a computer controller is provided to controls cement inflow parameters from a hydraulic source, the sensing system and energy delivery parameters for selectively heating tissue or polymerizing cement at both the interior and exterior of the injector to thereby control all parameters of cement injection to reduce workload on the physician.

In one embodiment, a lubricous surface layer is provided in the flow passageway of the bone cement injector to prevent sticking particularly when heating the cement.

In accordance with one embodiment, a medical apparatus for providing access to an interior portion of a bone is provided. The apparatus comprises an elongated sleeve configured for accessing a vertebral body, the sleeve defining a passageway extending from a proximal end to a distal open end of the sleeve, the sleeve having a first portion with a first cross-sectional area and a second portion with a second cross-sectional area, the second portion comprising threads about an exterior surface thereof and configured to engage bone, the second cross-sectional area being smaller than the first cross-sectional area. The apparatus also comprises a seal element extending across the passageway, the seal configured to allow the insertion of an instrument through the passageway and distal open end of the sleeve.

In accordance with another embodiment, a system for providing access to an interior portion of a vertebra is provided. The system comprises an elongated sleeve configured for accessing the interior of a vertebra, the sleeve having a handle end and a threaded distal end configured to threadably engage cortical bone of the vertebra, the sleeve defining a passageway extending from a proximal end to a distal open end thereof The system also comprises an elongated tool having a proximal end and sharp distal tip, the tool insertable through the passageway of the elongated sleeve and releasably lockable to the sleeve so that the distal tip extends outwardly of the distal open end of the sleeve by a selected dimension.

In accordance with still another embodiment, a method for treating a vertebral body is provided. The method comprises advancing an elongated sleeve through an incision in a patient's back to a vertebra, engaging the elongated sleeve with a pedicle of the vertebra and inserting at least one tool through the elongated sleeve and into the vertebral body to perform at least one medical procedure.

In accordance with yet another embodiment, a method for treating an abnormality in a bone is provided. The method comprises providing a first flow of an exothermic polymer into the interior of a bone, the first flow exhibiting a first level of polymerization, contemporaneously providing a second flow of a polymer into the bone, the second flow exhibiting a second level of polymerization, the first and second flows intermixing with each other, and allowing complete polymerization of the first and second flows to thereby provide a polymer bone cement with enhanced functional properties

These and other objects of the present invention will become readily apparent upon further review of the following drawings and specification.

### BRIEF DESCRIPTION OF THE DRAWINGS

In order to better understand the invention and to see how it may be carried out in practice, some preferred embodiments are next described, by way of non-limiting examples only, with reference to the accompanying drawings, in which like reference characters denote corresponding features consistently throughout similar embodiments in the attached drawings.

FIG. 1 is a schematic perspective view of a bone cement injection system in accordance with one embodiment.

FIG. 2 is another schematic perspective view of the bone cement injector of FIG. 1.

FIG. 3A is a schematic cross-sectional view of a vertebra showing one step in a method of injecting bone cement into a vertebra, in accordance with one embodiment.

FIG. 3B is a schematic cross-sectional view of the vertebra of FIG. 3A showing a subsequent step in said method for injecting bone into a vertebra.

FIG. 3C is a schematic cross-sectional view similar to FIGS. 3A-3B showing a subsequent step in said method of injecting bone into a vertebra.

FIG. 4 is a schematic perspective view of another embodiment of a bone cement injector.

FIG. 5 is a schematic sectional view of a distal portion of the bone cement injector of FIG. 4.

FIG. 6 is a schematic view of one embodiment of a pedicle port that can be used with the bone cement injectors of FIGS. 1-6.

FIG. 7A is a cut-away schematic view of a method step using the pedicle port of FIG. 6.

FIG. 7B is a schematic view of another step of a method of using the pedicle port of FIG. 6.

FIG. 7C is a schematic view of an additional step in using the pedicle port of FIG. 6.

FIG. 7D is a schematic view of another step in using the pedicle port of FIG. 6.

FIG. 7E is a schematic view of an additional step in using the pedicle port of FIG. 6.

FIG. 8 is a schematic perspective view of an another embodiment of a pedicle port.

### DETAILED DESCRIPTION OF THE PREFERRED EMBODIMENTS

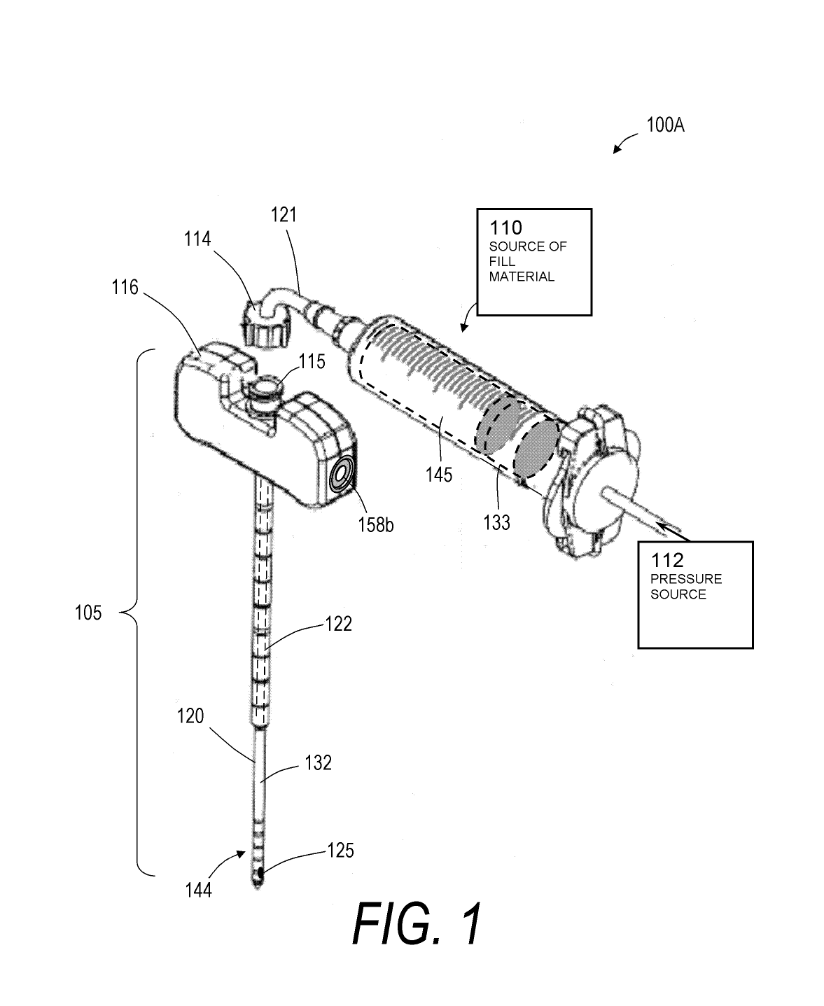

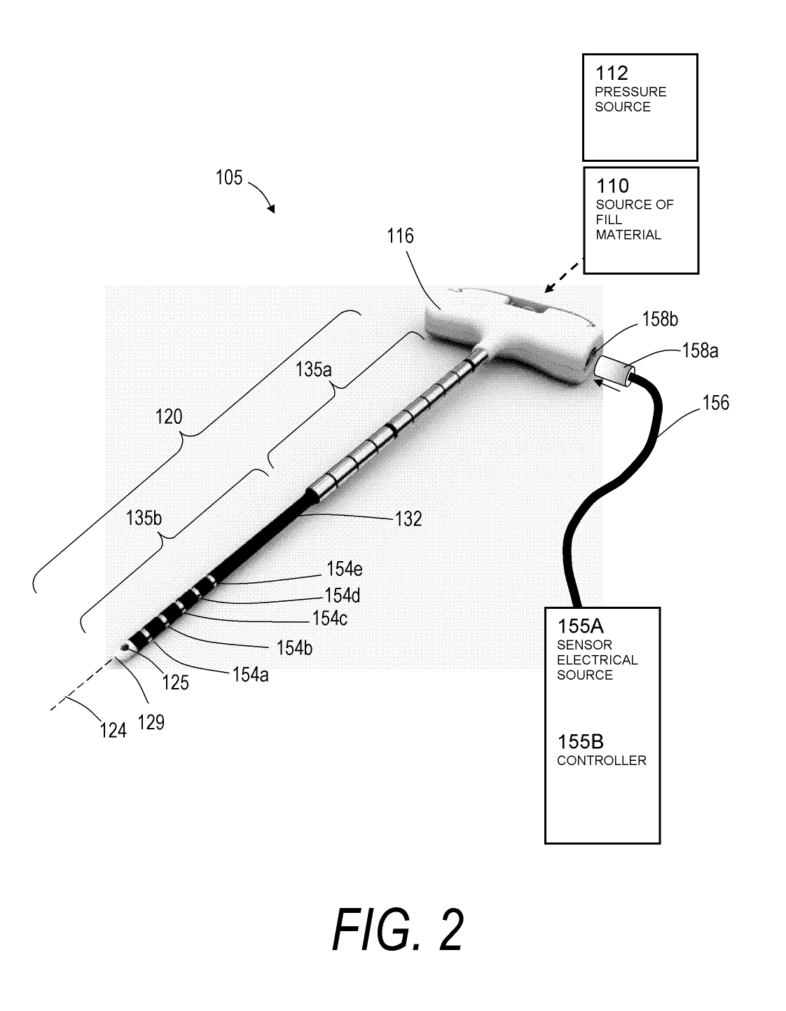

For the purposes of understanding the principles of the invention, reference will now be made to the embodiments illustrated in the drawings and accompanying text that describe the invention. Referring to FIGS. 1-2, one embodiment of bone fill introducer or injector system 100A is shown that can be used for treatment of the spine in a vertebroplasty procedure. The system 100A includes a bone cement injector 105 that is coupled to source 110 of a bone fill material wherein the injection of the fill material can be carried out by a pressure mechanism or source 112 operatively coupled to the source 110 of the bone fill material. In one embodiment as in FIG. 1, the pressure source 112 is a hydraulic actuator that is computer controlled, but the scope of the invention includes a manually operated syringe loaded with bone fill material, or any other pressurized source of fill material. The source 110 of fill material includes a coupling or fitting 114 for sealably locking to a cooperating fitting 115 at a proximal end or handle 116 of the bone cement injector 105 that has an elongated introducer sleeve indicated at 120. In the illustrated embodiment, the source 110 of bone fill material is a syringe-type source 110 coupled directly to fitting 115 with a flexible, rigid or bendable (deformable) hydraulic tube 121 extending toward the pressure source 112. The fill material then can flow through handle 116 to communicate with a passageway 122 in introducer sleeve 120.

As background, a vertebroplasty procedure using the invention would insert the introducer of FIG. 1 through a pedicle of a vertebra for accessing the osteoporotic cancellous bone. The initial aspects of the procedure are similar to a conventional percutaneous vertebroplasty wherein the patient is placed in a prone position on an operating table. The patient is typically under conscious sedation, although general anesthesia is an alternative. The physician injects a local anesthetic (e.g., 1% Lidocaine) into the region overlying the targeted pedicle or pedicles as well as the periosteum of the pedicle(s). Thereafter, the physician uses a scalpel to make a 1 to 5 mm skin incision over each targeted pedicle. Thereafter, the introducer is advanced through the pedicle into the anterior region of the vertebral body, which typically is the region of greatest compression and fracture. The physician confirms the introducer path posterior to the pedicle, through the pedicle and within the vertebral body by anteroposterior and lateral X-Ray projection fluoroscopic views. The introduction of infill material as described below can be imaged several times, or continuously, during the treatment depending on the imaging method.

### DEFINITIONS

“Bone fill, fill material, or infill material or composition” includes its ordinary meaning and is defined as any material for infilling a bone that includes an in-situ hardenable material or that can be infused with a hardenable material. The fill material also can include other “fillers” such as filaments, microspheres, powders, granular elements, flakes, chips, tubules and the like, autograft or allograft materials, as well as other chemicals, pharmacological agents or other bioactive agents.

“Flowable material” includes its ordinary meaning and is defined as a material continuum that is unable to withstand a static shear stress and responds with an irrecoverable flow (a fluid)-unlike an elastic material or elastomer that responds to shear stress with a recoverable deformation. Flowable material includes fill material or composites that include a fluid (first) component and an elastic or inelastic material (second) component that responds to stress with a flow, no matter the proportions of the first and second component, and wherein the above shear test does not apply to the second component alone.

“Substantially” or “substantial” mean largely but not entirely. For example, substantially may mean about 10% to about 99.999%, about 25% to about 99.999% or about 50% to about 99.999%.

“Osteoplasty” includes its ordinary meaning and means any procedure wherein fill material is delivered into the interior of a bone.

“Vertebroplasty” includes its ordinary meaning and means any procedure wherein fill material is delivered into the interior of a vertebra.

In FIGS. 1-5, it can be seen that elongated introducer sleeve 120 of bone cement injector 105 includes the interior channel 122 extending about axis 124, wherein the channel 122 terminates in a distal outlet opening 125. The outlet opening 125 can be a single opening or a plurality of openings about the radially outward surface 128 of sleeve 120 or an opening at the distal tip 129 the sleeve. The distal tip 129 can be blunt or sharp. In one embodiment, a core portion 130 of sleeve 120 is an electrically conductive metal sleeve, such as a stainless steel hypo tube. The core sleeve portion 130 has both an exterior insulative coating 132 and an interior insulative coating that will be described in greater detail below.

In one embodiment as shown in FIGS. 1-2, the bone fill system 100A has a container of fill material source 110 that is pressurized by a hydraulic source acting on a floating piston 133 (phantom view) in the syringe-like source 110 that carries the fill material. In FIGS. 1-2, it can be seen introducer sleeve 120 has a proximal portion 135a that is larger in cross-section than distal portion 135b with corresponding larger and smaller interior channel portions therein. This allows for lower injection pressures since the cement flow needs to travel less distance through the smallest diameter distal portion of the introducer sleeve. The distal portion 135b of the introducer can have a cross-section ranging between about 2 mm and 4 mm with a length ranging between about 40 mm and 60 mm. The proximal portion 135a of introducer sleeve 120 can have a cross-section ranging between about 5 mm and 15 mm, or between about 6 mm and 12 mm.

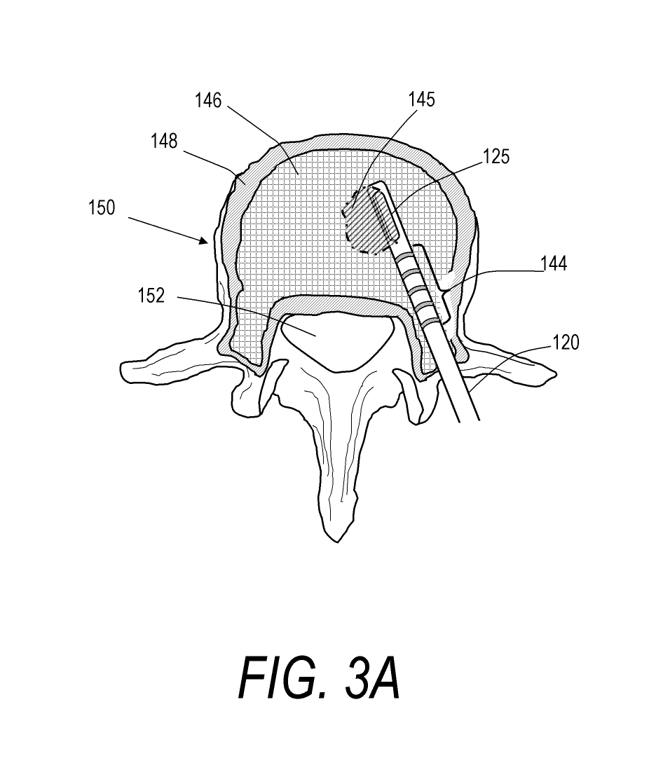

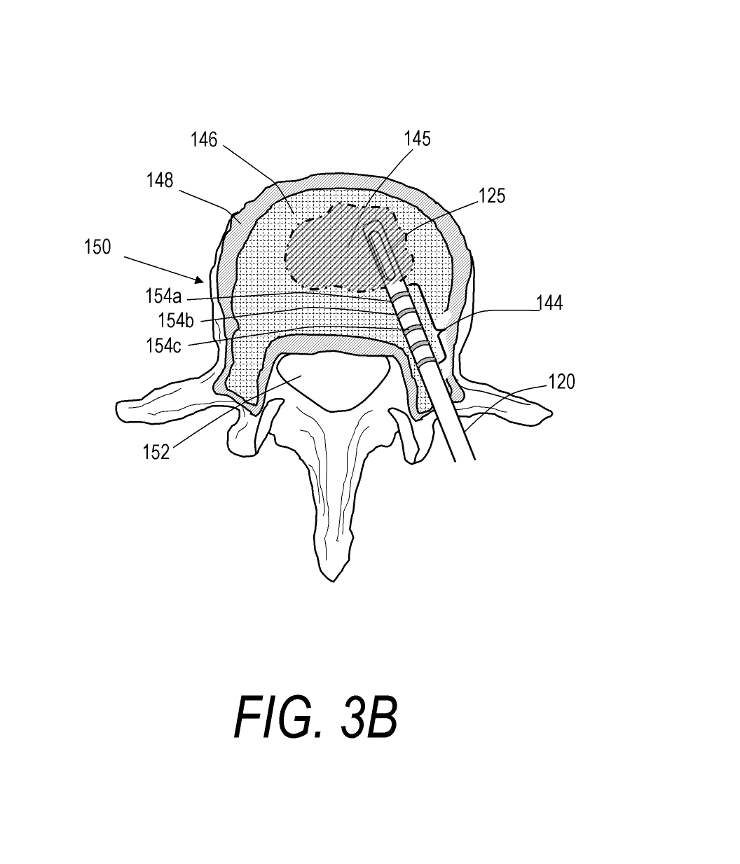

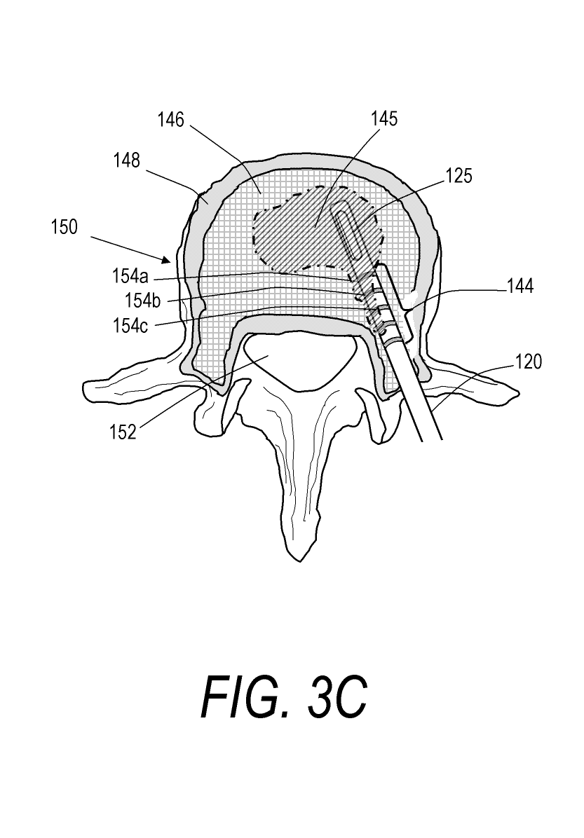

As can be seen in the embodiment illustrated in FIGS. 1-2, the exterior surface of introducer sleeve 120 carries a sensor system indicated at 144 that can sense the flow or movement of a fill material or cement 145 (see FIGS. 3A-3C) proximate to the sensor system 144. The introducer sleeve 120 with such a sensor system 144 is particularly useful in monitoring and preventing extravasation of fill material 145 in a vertbroplasty procedure.

In one embodiment and method of use, referring to FIGS. 3A-3C, the introducer sleeve 120 is used in a conventional vertebroplasty with a single pedicular access or a bi-pedicular access. The fill material 145 can be a bone cement, such as PMMA, that is injected into cancellous bone 146 which is within the interior of the cortical bone surface 148 of vertebra 150.

In FIGS. 3A-3B, it can be seen that a progressive flow of cement 145 is provided from outlet 125 of introducer sleeve 120 into the interior of the vertebra 150. FIG. 3A illustrates an initial flow volume with FIG. 3B illustrating an increased flow volume of cement 145. FIG. 3C depicts a situation that is known to occur wherein bone is fractured along the entry path of introducer 120 wherein the cement 145 under high injection pressures finds the path of least resistance to be at least partly in a retrograde direction along the surface of introducer 120. The retrograde flow of cement as in FIG. 3C, if allowed to continue, could lead to cement extravasation into the spinal canal 152 which can lead to serious complications. As can be understood from FIG. 3C, the sensor system 144 can be actuated when cement 145 comes into contact with the sensor system 144. In one embodiment as shown in FIGS. 2-3C, the sensor system 144 comprises a plurality of spaced apart exposed electrodes or electrode portions (e.g., electrodes 154a, 154b, 154c etc.) coupled to sensor electrical source 155A via cable 156 and plug 158a connected to electrical connector 158b in the proximal handle end 116 of the introducer 120, wherein the electrical source carries a low voltage direct current or Rf current between the opposing potentials of spaced apart electrodes. The voltage can be from about 0.1 volt to 500 volts, or from about 1 volt to 5 volts and will create a current path through the tissue between a pair of electrodes. The current can be continuous, intermittent and/or multiplexed between different electrode pairs or groups of electrodes. The arrangement of electrodes can be spaced apart ring-type electrodes and axially spaced apart as shown in FIGS. 1 and 2, or the electrodes can be discrete elements, helically spaced electrodes, or the electrodes can be miniaturized electrodes as in thermocouples, MEMS devices or any combination thereof The number of sensors or electrodes can range from about 1 to 100 and can be adapted to cooperate with a ground pad or other surface portion of sleeve 120. In one embodiment, the electrodes can include a PTC or NTC material (positive temperature coefficient of resistance or negative temperature coefficient of resistance) to thereby function as a thermistor to allow measurement of temperature as well as functioning as a sensor. The sensor system 144 includes a controller 155B (FIG. 2) that can measure at least one selected parameter of the current flow to determine a change in a parameter (e.g., impedance). When the non-conductive bone cement 145 contacts one or more electrodes of the sensor system 144, the controller 155B identifies a change in the selected electrical parameter and generates a signal to the operator. The scope of the invention includes sensor systems capable of sensing a change in electrical properties, reflectance, fluorescence, magnetic properties, chemical properties, mechanical properties or a combination thereof

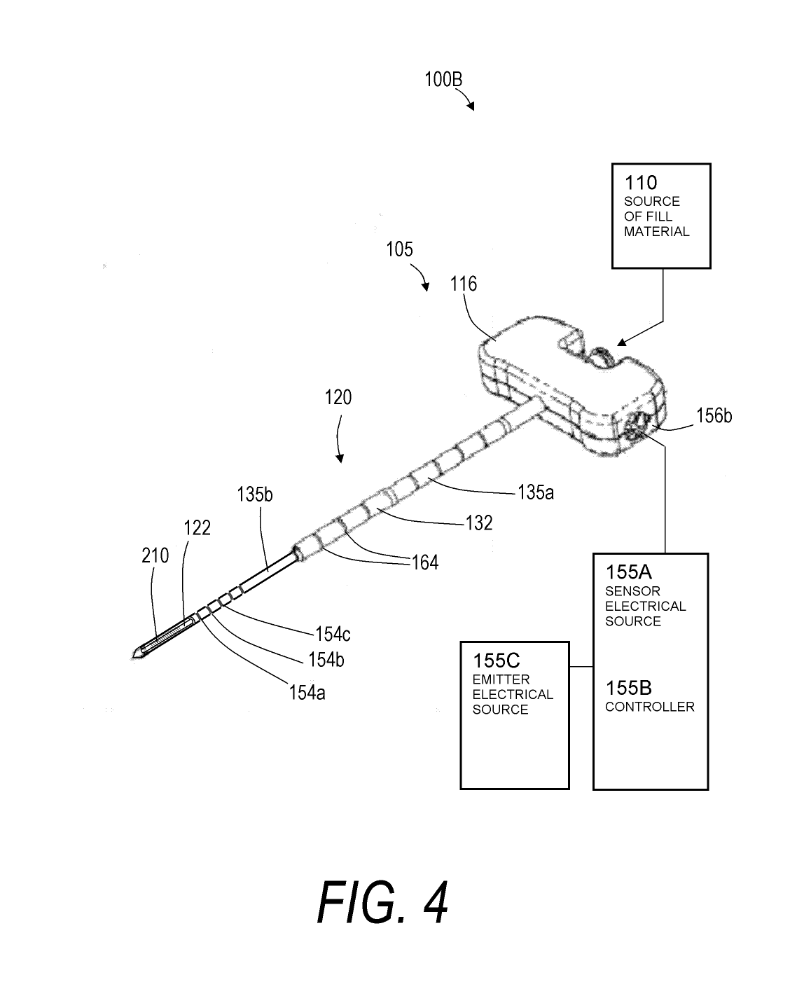

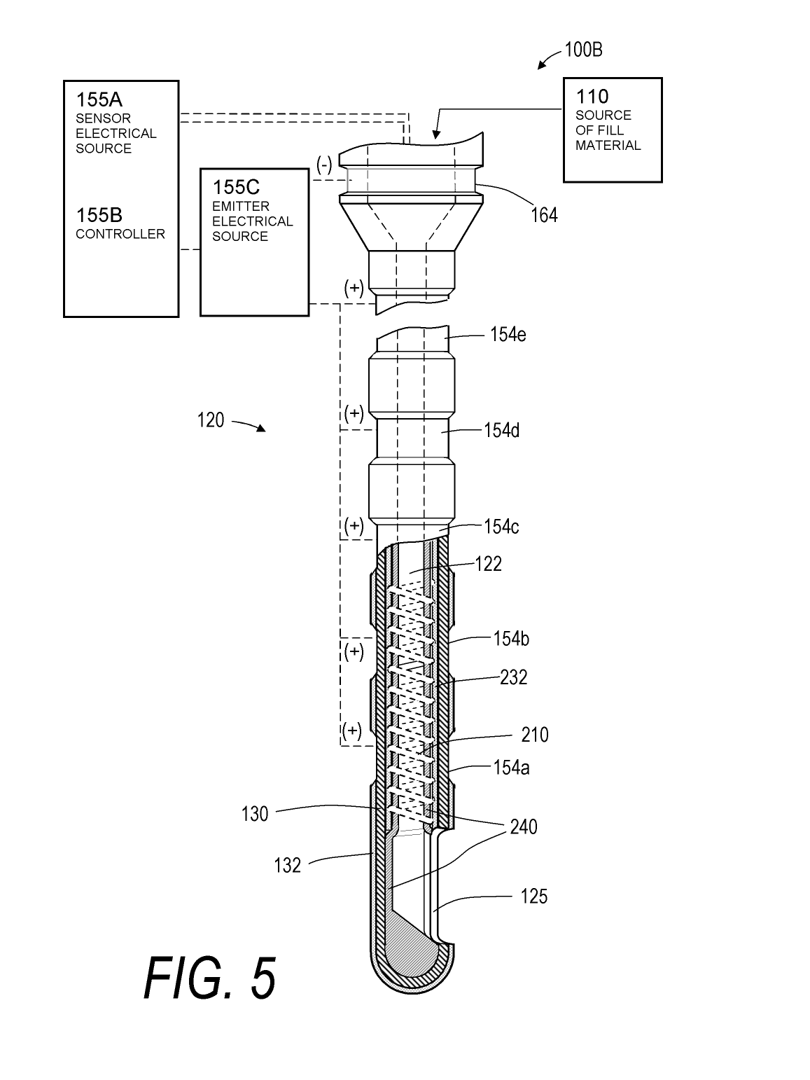

Now referring to FIGS. 4 and 5, an alternative system 100B includes bone cement injector 105 that is similar to the injector of FIGS. 1-2, but with a different embodiment of sensor system together with an additional electrical energy delivery system for applying energy to fill material for altering its viscosity. In this embodiment, the ring electrode portions (i.e. electrodes 154a, 154b, 154c, etc. in phantom view) are exposed portions of a metal core portion 130 of sleeve 120 (see FIG. 5) that is coupled via lead 156b to electrical source 155C. The electrode portions 154a, 154b, 154c are indicated having a first polarity (+) that cooperate with one or more second polarity (−) return electrodes 164 in a more proximal portion of the sleeve coupled by lead 156b to sensor electrical source 155A. In this embodiment, current flows through the multiple electrode portions 154a, 154b, 154c and then though engaged tissue to the return electrodes 164, wherein the current flow will signal certain impedance parameters before and during an initial injection of cement 145, as in FIGS. 3A-3B. When there is a retrograde flow of cement 145 as in FIG. 3C that covers one or more electrode portions 154a, 154b, 154c, then the electrical parameter (e.g., impedance) changes to thereby signal the operator that such a retrograde flow has contacted or covered an electrode portion 154a, 154b or 154c. The change in parameter can be a rate of change in impedance, a change in impedance compared to a data library, etc. which will signal the operator of such a flow, wherein the controller 155B can operate to automatically terminate the activation of pressure source 112 to cease continued injection of bone fill material.

In the system of FIGS. 4 and 5, the bone fill injection system further includes a thermal energy emitter within an interior channel 122 of the introducer 120 for heating a flow of bone cement from an open termination 125 in the introducer 120. In one embodiment, the thermal energy emitter is a resistive heating element 210 that can elevate the temperature of cement 145 to at least 50° C., at least 60° C., at least 70° C. or at least 80° C. The resistive element 210 is coupled to emitter electrical source 155C, as depicted in FIGS. 4 and 5, together with controller 155B, where the controller 155B can control cement inflow parameters such as variable flow rates, constant flow rates and/or pulsed flows in combination with controlled energy delivery. The thermal energy delivery is adapted to accelerate polymerization and increase the viscosity of a PMMA or similar bone cement as disclosed in the co-pending U.S. patent applications listed below. In another embodiment, the thermal energy emitter can be an Rf emitter for ohmically heating a bone cement that carries electrically conductive compositions as disclosed in the below co-pending U.S. patent applications Ser. No. 11/165,652 filed Jun. 24, 2005; Ser. No. 11/165,651 filed Jun. 24, 2005; Ser. No. 11/208,448 filed Aug. 20, 2005; and Ser. No. 11/209,035 filed Aug. 22, 2005. In another embodiment, the thermal energy emitter for delivering thermal energy to bone cement can be selected from the group consisting of a resistively heated emitter, a light energy emitter, an inductive heating emitter, an ultrasound source, a microwave emitter and any other electromagnetic energy emitter to cooperates with the bone cement. In FIGS. 4 and 5, the controller 155B can control (i) heating of the bone cement, (ii) the cement injection pressure and/or flow rate, (iii) energy delivery to cement flows in or proximate the distal end of the introducer and (iv) energy delivery to sense retrograde flows about the exterior surface of the introducer.

In one embodiment depicted in FIG. 5, the resistive heating element 210 comprises a helically wound coil of a resistive material in interior bore 122 of the introducer 120. The heating element 210 optionally is further formed from, or coated with, a positive temperature coefficient material and coupled to a suitable voltage source to provide a constant temperature heater as is known in the art. As can be seen in FIG. 5, the heating element 210 is carried within insulative coating 232 in the interior of core sleeve 130 which is a conductive metal as described above.

Another aspect of the invention can be understood from FIG. 5, where it can be seen that the exterior surface of sleeve 120 has an insulative, scratch-resistant coating indicated at 132 that comprises a thin layer of an insulative amorphous diamond-like carbon (DLC) or a diamond-like nanocomposite (DCN). It has been found that such coatings have high scratch resistance, as well as lubricious and non-stick characteristics that are useful in bone cement injectors of the invention. Such coatings are particularly useful for an introducer sleeve 120 configured for carrying electrical current for (i) impedance sensing purposes; (ii) for energy delivery to bone fill material; and/or (iii) ohmic heating of tissue. For example, when inserting a bone cement injector through the cortical bone surface of a pedicle and then into the interior of a vertebra, it is important that the exterior insulative coating portions do not fracture, chip or scratch to thereby insure that the electrical current carrying functions of the injector are not compromised.

The amorphous diamond-like carbon coatings and the diamond-like nanocomposites are available from Bekaert Progressive Composites Corporations, 2455 Ash Street, Vista, Calif. 92081 or its parent company or affiliates. Further information on the coating can be found at: http://www.bekaert.com/bac/Products/Diamond-like%20coatings.htm, the contents of which are incorporated herein by reference. The diamond-like coatings comprise amorphous carbon-based coatings with high hardness and low coefficient of friction. The amorphous carbon coatings exhibit non-stick characteristics and excellent wear resistance. The coatings are thin, chemically inert and have a very low surface roughness. In one embodiment, the coatings have a thickness ranging between 0.001 mm and 0.010 mm; or between 0.002 mm and 0.005 mm. The diamond-like carbon coatings are a composite of sp2 and sp3 bonded carbon atoms with a hydrogen concentration between 0 and 80%. Another diamond-like nanocomposite coatings (a-C:H/a-Si:O; DLN) is made by Bakaert and is suitable for use in the bone cement injector of the invention. The materials and coatings are known by the names Dylyn®Plus, Dylyn®/DLC and Cavidur®.

FIG. 5 further illustrates another aspect of bone cement injector 105 that again relates to the thermal energy emitter (resistive heater 210) within interior passageway 122 of introducer 120. In one embodiment, it has been found that it is advantageous to provide a lubricious surface layer 240 within the interior of resistive heater 210 to insure uninterrupted cements flows through the thermal emitter without sticking. In one embodiment, surface layer 240 is a fluorinated polymer such as Teflon® or polytetrafluroethylene (PTFE). Other suitable fluoropolymer resins can be used such as FEP and PFA. Other materials also can be used such as FEP (Fluorinated ethylenepropylene), ECTFE (Ethylenechlorotrifluoroethylene), ETFE, Polyethylene, Polyamide, PVDF, Polyvinyl chloride and silicone. The scope of the invention includes providing a bone cement injector having a flow channel extending therethrough with at least one open termination 125, wherein a surface layer 240 within the flow channel has a static coefficient of friction of less than 0.5, less than 0.2, or less than 0.1.

In another embodiment, the bone cement injector has a flow channel 122 extending therethrough with at least one open termination 125, wherein at least a portion of the surface layer 240 of the flow channel is ultrahydrophobic or hydrophobic which may better prevent a hydrophilic cement from sticking.

In another embodiment, the bone cement injector has a flow channel 122 extending therethrough with at least one open termination 125, wherein at least a portion of the surface layer 240 of the flow channel is hydrophilic for which may prevent a hydrophobic cement from sticking.

In another embodiment, the bone cement injector has a flow channel 122 extending therethrough with at least one open termination in a distal end thereof, wherein the surface layer 240 of the flow channel has high dielectric strength, a low dissipation factor, and/or a high surface resistivity.

In another embodiment, the bone cement injector has a flow channel 122 extending therethrough with at least one open termination 125 in a distal end thereof, wherein the surface layer 240 of the flow channel is oleophobic. In another embodiment, the bone cement injector has a flow channel 122 extending therethrough with at least one open termination 125 in a distal end thereof, wherein the surface layer 240 of the flow channel has a substantially low coefficient of friction polymer or ceramic.

In another embodiment, the bone cement injector has a flow channel 122 extending therethrough with at least one open termination 125 in a distal end thereof, wherein the surface layer 240 of the flow channel has a wetting contact angle greater than 70°, greater than 85°, and greater than 100°.

In another embodiment, the bone cement injector has a flow channel 122 extending therethrough with at least one open termination in a distal end thereof, wherein the surface layer 240 of the flow channel has an adhesive energy of less than 100 dynes/cm, less than 75 dynes/cm, and less than 50 dynes/cm.

The apparatus above also can be configured with any other form of thermal energy emitter that includes the non-stick and/or lubricious surface layer as described above. In one embodiment, the thermal energy emitter can comprise at least in part an electrically conductive polymeric layer. In one such embodiment, the electrically conductive polymeric layer has a positive temperature coefficient of resistance.

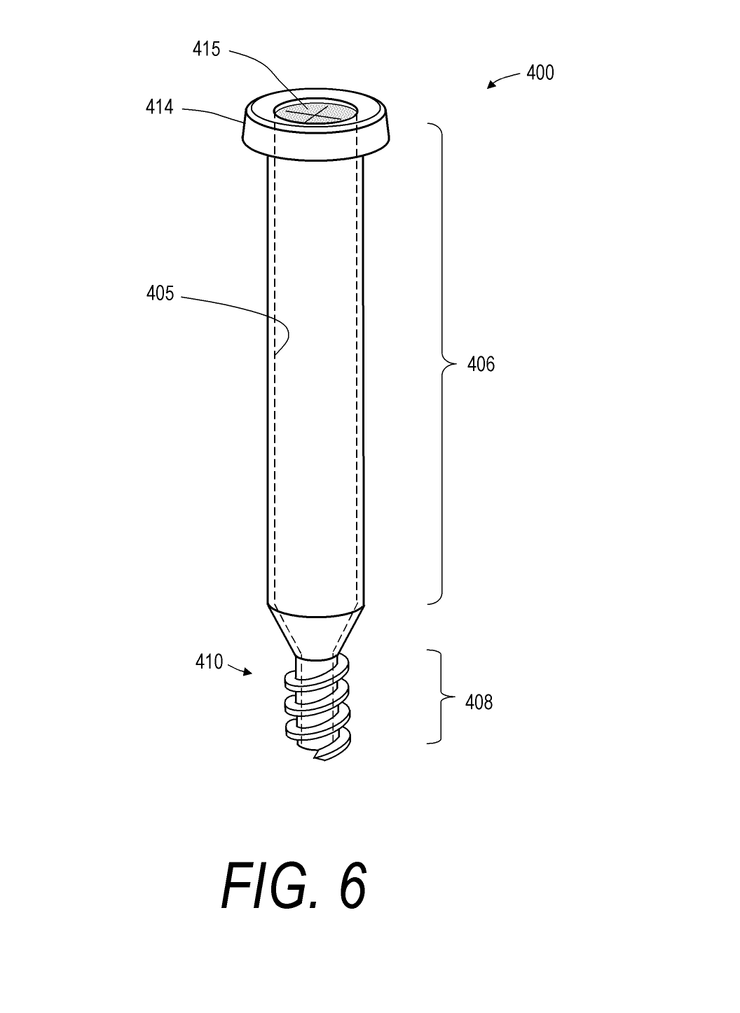

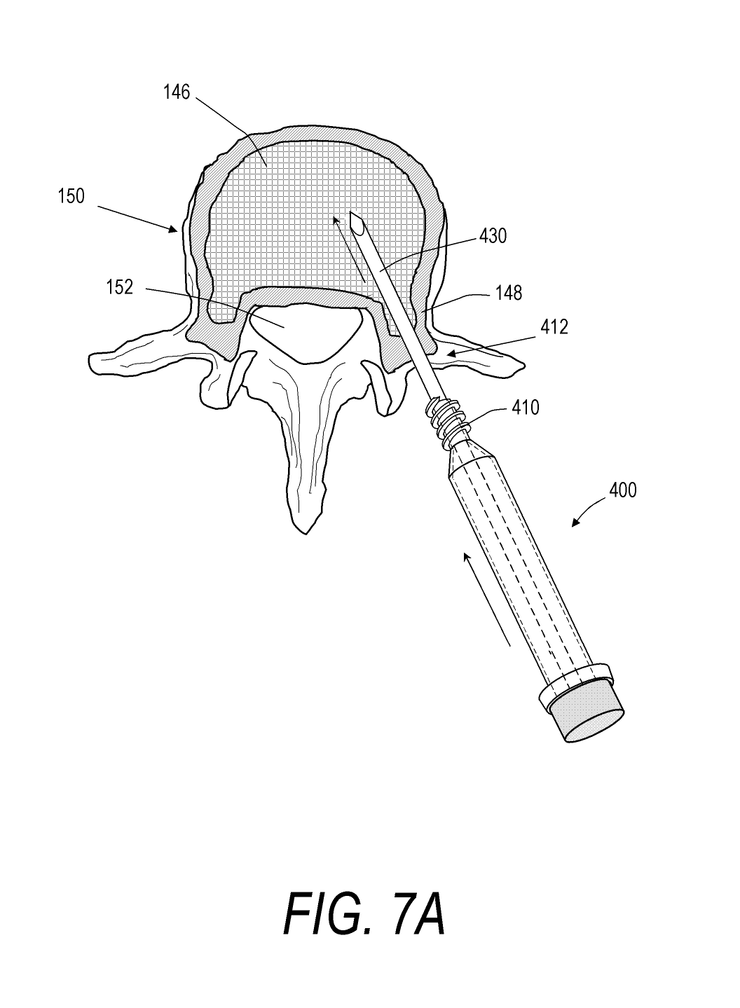

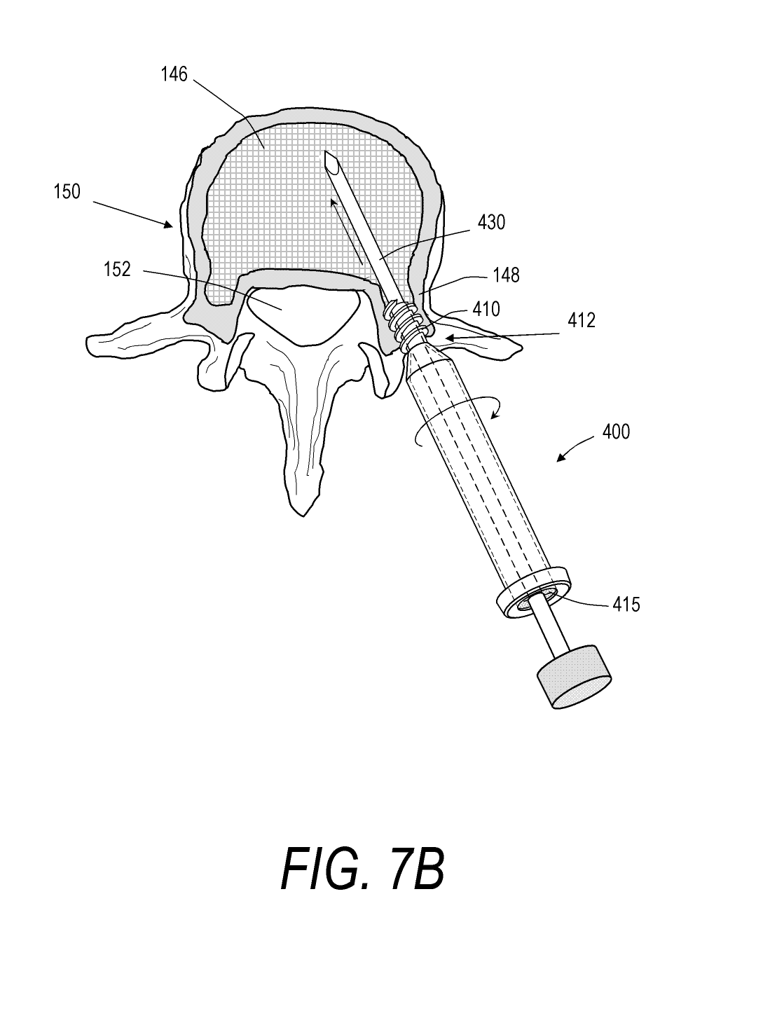

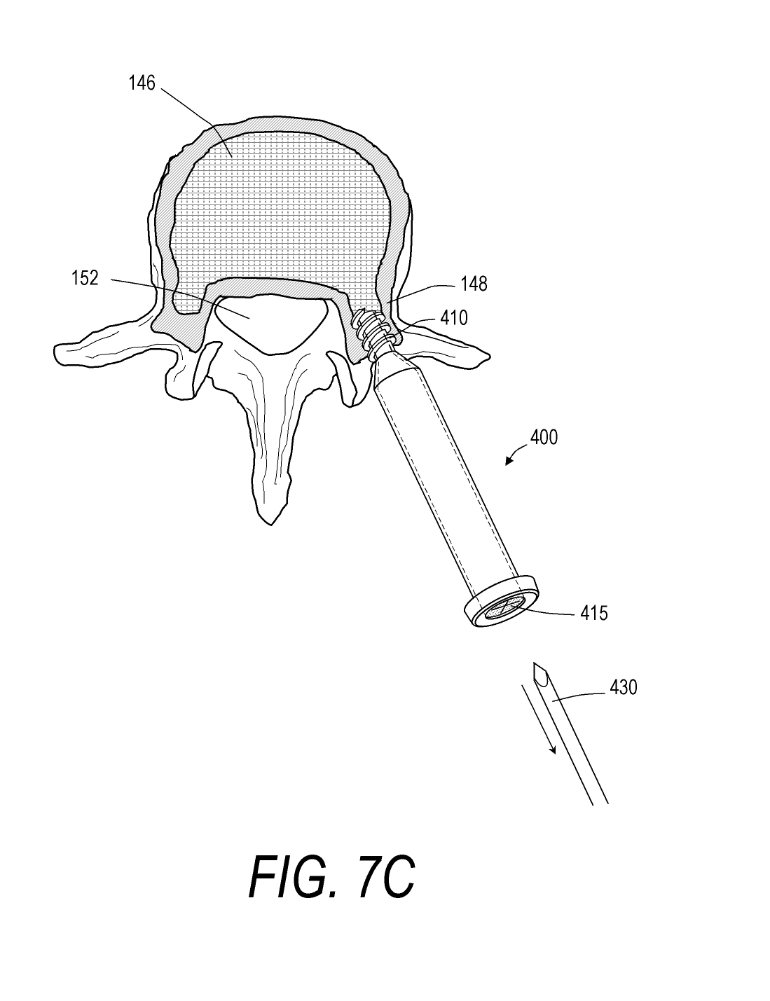

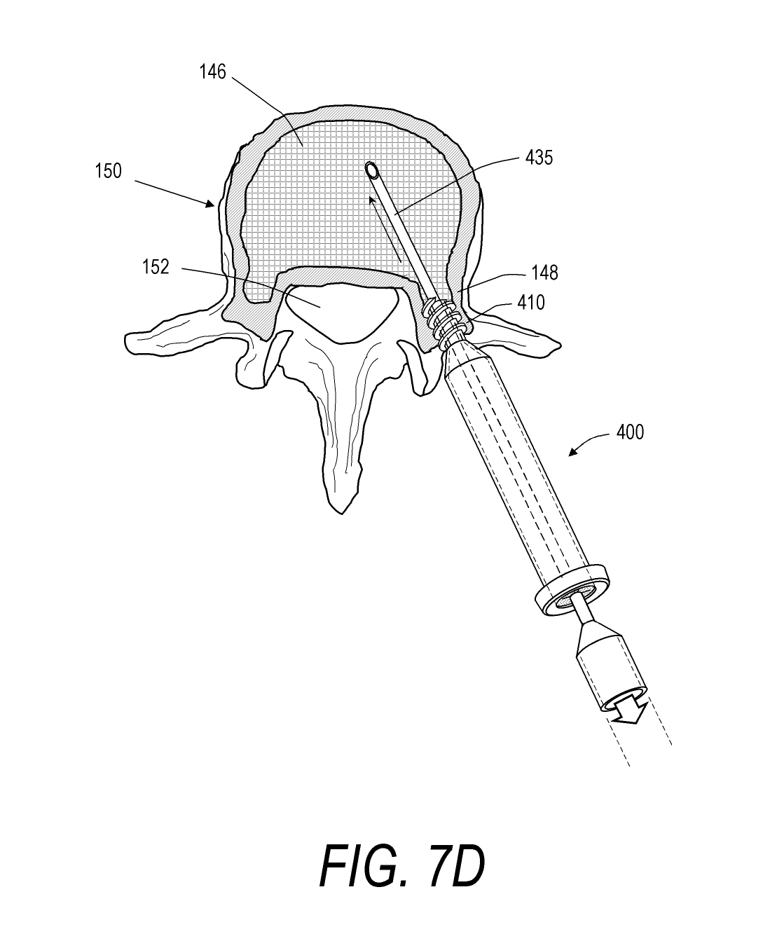

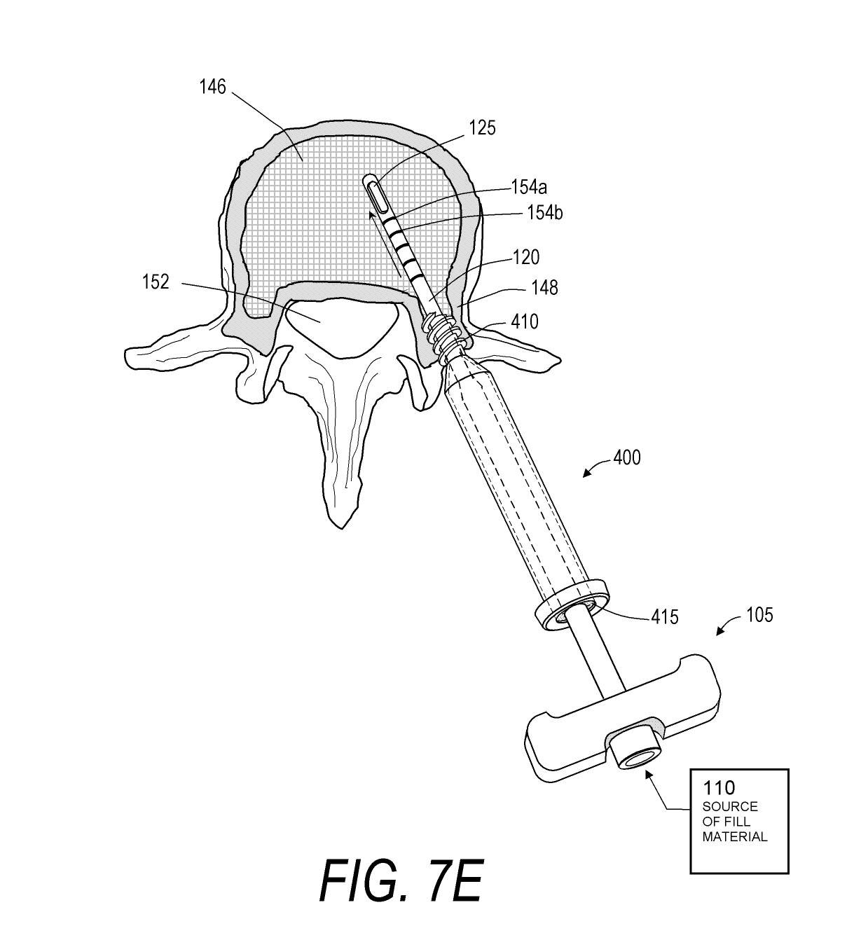

FIG. 6 is an illustration of another spine treatment apparatus that may be described as a pedicle port 400. The port 400 can be thin-walled with a passageway 405 extending therethrough to allow the insertion and withdrawal or various instruments from the interior of a vertebra 150 (see FIG. 7A-7E). The port 400 can be a tubular sleeve having a first (proximal) sleeve portion 406 and a second (distal) sleeve portion 408. The second sleeve portion 408 can be threaded and in one embodiment can have helical threads 410 for engaging the cortical bone surface 148 of a targeted pedicle 412 (see FIGS. 7A-7B). The threads can be self-tapping threads, and can be tapered with cutting edges as in known in the art.

As can be seen in FIG. 6, the proximal end of port 400 can have a grip portion indicated at 414 that can be, for example, a flange or handle suitable for gripping with the operator's fingers to facilitate handling the port 400 (e.g., to screw the port 400 into bone) as will be described below. The port 400 can further include a seal element 415 that can be an elastomeric or flexible member as in known in the art with one or more cuts to allow the insertion of instrument or tools through the interior passageway 405. The seal 415 also can be a hinged flap-type seal and the seal can be positioned in a proximal, medial or distal portion of proximal port portion 406. Advantageously, the seal 415 can allow insertion of tools through the port 400 into the vertebra 150, and substantially inhibit foreign matter from entering the port 400.

In one embodiment of port 400 shown in FIG. 6, the cross-section of the proximal port portion 406 has a mean diameter ranging from 5 mm to 40 mm, 8 mm to 25 mm, or 10 mm to 15 mm. The port 400 has distal port portion 408 with a cross-section of less that about 8 mm, or less that about 6 mm. The distal port portion 408 has an axial length suitable for engaging cortical bone, which axial length can range between 2 mm and 15 mm, and between 4 mm and 10 mm. In one embodiment, the port can be of metal and can have an electrically insulative coating.

FIGS. 7A-7E illustrate a method of using the pedicle port 400 of FIG. 6, wherein FIG. 7A first illustrates a sharp-tipped trocar 430 being inserted through the port 400 and advanced through the pedicle 412 into the interior of the vertebra 150. FIG. 7B next illustrates the port 400 being advanced distally and screwed into the cortical bone 148 of the pedicle 412. FIG. 7C next illustrates the trocar 430 being withdrawn from the port 400. FIG. 7D illustrates another medical tool 435 used for treatment or diagnosis being inserted through the port 400. More particularly in FIG. 7D, a biopsy needle as known in the art is shown wherein aspiration forces (indicated by the arrow) can be applied to withdrawal of matter from the interior of the vertebra 150. It should be appreciated that any tool such as an electrosurgical tool, an ultrasound tool, a needle for injecting therapeutics or any other sort of tool can be inserted safely through the port 400.

FIG. 7E illustrates a bone cement injector 105 as in FIGS. 4-5 being inserted through the port 400 to deliver bone cement as described above with reference to FIGS. 3A-3C. It has been found that the port 400 is particularly useful because it allows withdrawal of the injector sleeve 120 through the passageway in the port 400 so that the sleeve working end does not come in contact with soft tissue outside the vertebra. In the absence of a pedicle port 400, it is possible that a small amount of bone cement 145 might leak from inflow port 125 and contact tissue which can cause an irritation, as can be understood from FIG. 3A. The pedicle port 400 therefore provides a safe passageway for inserting and removing tools from a vertebra—as well as preventing injected material from contacting soft tissue outside the vertebra.

In another embodiment of the pedicle port 400, the surface of the sleeve can be electrically insulated with suitable materials, such as those described above. In another embodiment, at least the distal portion of the pedicle port 400 can be electrically conductive to cooperate with the return electrode 164 on the exterior of the bone cement injector sleeve 105 as in FIGS. 4-5 to thus allow the sensing system to function as described above.

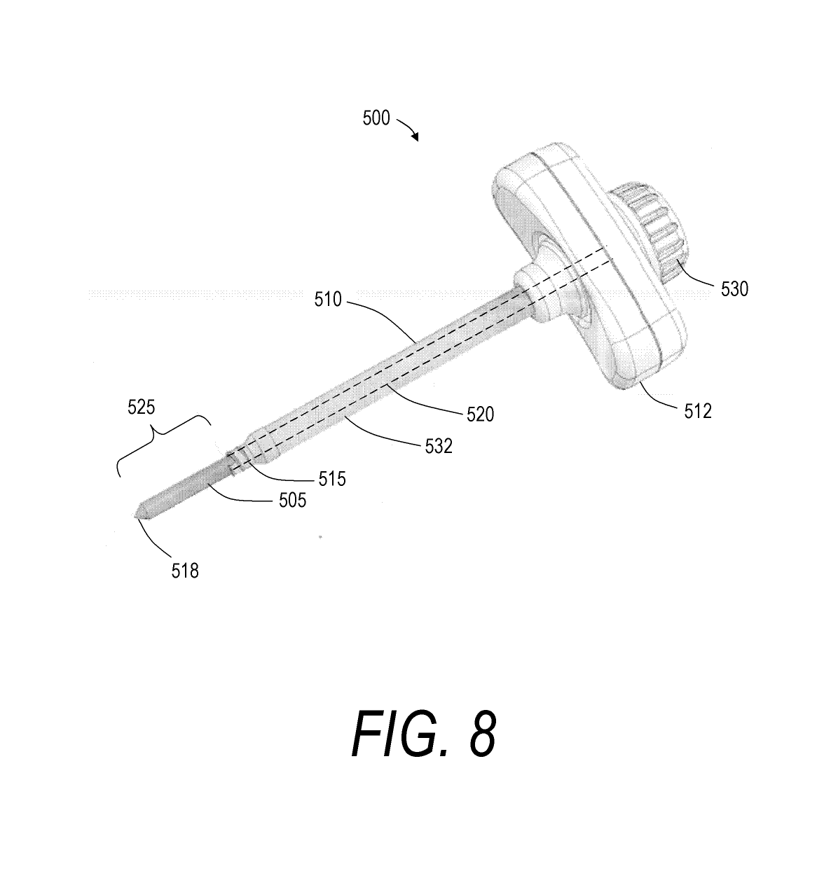

FIG. 8 depicts another embodiment of a pedicle port 500 that can form a kit or assembly with a penetrating tool 505 that is releasably lockable at a certain position within the interior passageway of the sleeve 510 of the port 500. Again, the elongated tubular sleeve 510 can be used for accessing the interior of a vertebra and the port 500 has a handle end 512 and a threaded distal end 515 for threaded engagement of cortical bone. The penetrating tool 505 can have a sharp distal tip 518 and the tool shaft 520 can be releaseable locked in a desired position in the interior passageway of the sleeve 510, with the sharp distal tip 518 extending outwardly a fixed selected dimension 525 from the threaded distal end 515 of the sleeve 510. In one embodiment, the selected dimension 525 is fixed, but also can be adjustable between a plurality of fixed dimensions.

In FIG. 8, the selected dimension 525 is less than 30 mm, 20 mm and 15 mm. In another embodiment, the selected dimension ranges from 1 mm to 30 mm, 1 mm to 20 mm and 1 mm to 15 mm.

As depicted in FIG. 8, one embodiment includes a locking mechanism for locking the handle end 512 of the sleeve and the proximal end 530 of the tool 505 at the proximal end of the assembly. In the embodiment illustrated in FIG. 8, the locking mechanism includes mating thread elements. In another embodiment, the locking mechanism can include a cam element, a cam sleeve, or holes or grooves or notch features in the tool shaft 510 or end 530 that engage a corresponding pin, ratchet or the like.

In one embodiment as in FIG. 8, the handle end 512 includes a flexible seal in the interior passageway, such as the seal discussed above. In another embodiment, the handle end 512 can be transparent and include a flap-type seal in the interior passageway in the handle end 512 as is known in the art.

In one embodiment as in FIG. 8, the sharp distal tip 518 of the tool is at least one of beveled, faceted and conical.

In another embodiment as in FIG. 8, the sleeve includes at least one lumen in a wall 532 of the sleeve that can be coupled to an irrigation and/or irrigation source.

In one embodiment of a method for treating bone, an elongated tubular sleeve is provided for posterior pedicular access to a vertebra, the sleeve having a handle end and a threaded distal end. The method also includes threadably engaging the threaded distal end of the sleeve with cortical bone of a pedicular penetration, and performing at least one medical procedure with a tool inserted through the sleeve. The initial steps of the method include making an incision to access the pedicle and penetrating the cortical bone of the pedicle with the sharp-tipped tool.

In another aspect of the method, the penetrating step can be accomplished with a sharp-tipped tool inserted through the tubular sleeve, and the penetrating step can include limiting the depth of penetration of the sharp-tipped tool. For example, the penetrating step can include limiting the depth of penetration of the sharp-tipped tool to the posterior ⅔ of the vertebral body, the posterior ½ of the vertebral body and the posterior ⅓ of the vertebral body.

In another aspect of a method for treating a vertebral body, medical procedures can be performed selected from the group of bone cement injection; obtaining tissue or fluid; a step in performing a biopsy; treating a tumor with at least one of thermal energy delivery, cryogenic energy delivery, pharmacological energy delivery, mechanical energy delivery, and the implantation of radiation emitting seeds; creating a space in vertebral cancellous bone; creating a space in vertebral cancellous bone by means of tamping, cutting, drilling, abrading, breaking and fracturing; creating a space in vertebral cancellous bone by expanding an expandable member; creating a space in vertebral cancellous bone by expanding an expandable member with a fluid introduced into an interior chamber of the expandable member; creating a space in vertebral cancellous bone by expanding a stent; creating a space in vertebral cancellous bone by rotating an expandable member; creating a space in vertebral cancellous bone by vibrating a member; creating a space in vertebral cancellous bone by ultrasonically actuating a member; irrigating the cancellous bone; aspirating at least one of tissue and fluids; or creating a space in vertebral cancellous bone by multiple axial translations of a working end of the tool.

In another method corresponding to the invention, it has been found that applying energy to bone cement flows proximate to the inflow port of an injector can greatly improve certain physical properties of the cement when fully cured. In one embodiment, it appears that energy delivery to the inflowing cement can more greatly polymerize a surface portion of the flow when compared to interior portions of the flow. Thereafter, the more polymerized portions intermix with the less polymerized portions as the flows are disrupted by flowing into cancellous bone. Upon complete curing of the bone cement volume, the fragmented, more polymerized portions apparently function as reinforcing fibers within the cement. It has been found that fatigue life of the bone cement can be increased by 5×, 10× or even 20×.

This aspect of the invention includes enhancing a functional physical property of a polymer bone cement, with the method including providing a first flow of an exothermic polymer through an injector into the interior of a bone wherein the first flow exhibits a first level of polymerization, and contemporaneously providing a second flow of a polymer through the injector wherein the second flow exhibits a second level of polymerization wherein the first and second flows intermix to increase fatigue life. The method also includes enhancing a physical property of the cement such as bending strength, compressive strength and tensile strength. The method can apply thermal energy to the second flow by means of at least one of applying light energy, applying thermal energy from a resistive heating element, applying thermal energy from a heated vapor media, applying thermal energy from a radiofrequency source, applying thermal energy from a microwave source and applying thermal energy from an ultrasound source.

In another embodiment, the step of applying thermal energy is accomplished by a resistive heating element that includes a positive temperature coefficient of resistance (PTCR) material.

In another embodiment, the step applying thermal energy is accomplished by light energy from an LED, or from at least one of coherent light and non-coherent light.

In related methods of the invention, the system of the invention can use any suitable energy source, other that radiofrequency energy, to accomplish the purpose of altering the viscosity of the fill material 145. The method of altering fill material can be at least one of a radiofrequency source, a laser source, a microwave source, a magnetic source and an ultrasound source. Each of these energy sources can be configured to preferentially deliver energy to a cooperating, energy sensitive filler component carried by the fill material. For example, such filler can be suitable chromophores for cooperating with a light source, ferromagnetic materials for cooperating with magnetic inductive heating means, or fluids that thermally respond to microwave energy.

The scope of the invention includes using additional filler materials such as porous scaffold elements and materials for allowing or accelerating bone ingrowth. In any embodiment, the filler material can comprise reticulated or porous elements of the types disclosed in co-pending U.S. patent application Ser. No. 11/146,891, filed Jun. 7, 2005, titled “Implants and Methods for Treating Bone” which is incorporated herein by reference in its entirety and should be considered a part of this specification. Such fillers also can carry bioactive agents. Additional fillers, or the conductive filler, also can include thermally insulative solid or hollow microspheres of a glass or other material for reducing heat transfer to bone from the exothermic reaction in a typical bone cement component.

The above description of the invention is intended to be illustrative and not exhaustive. Particular characteristics, features, dimensions and the like that are presented in dependent claims can be combined and fall within the scope of the invention. The invention also encompasses embodiments as if dependent claims were alternatively written in a multiple dependent claim format with reference to other independent claims. Specific characteristics and features of the invention and its method are described in relation to some figures and not in others, and this is for convenience only. While the principles of the invention have been made clear in the descriptions and combinations included herein, it will be obvious to those skilled in the art that modifications may be utilized in the practice of the invention, and otherwise, which are particularly adapted to specific environments and operative requirements without departing from the principles of the invention. The appended claims are intended to cover and embrace any and all such modifications, with the limits only of the true purview, spirit and scope of the invention.

Of course, the foregoing description is that of certain features, aspects and advantages of the present invention, to which various changes and modifications can be made without departing from the spirit and scope of the present invention. Moreover, the bone treatment systems and methods need not feature all of the objects, advantages, features and aspects discussed above. Thus, for example, those skill in the art will recognize that the invention can be embodied or carried out in a manner that achieves or optimizes one advantage or a group of advantages as taught herein without necessarily achieving other objects or advantages as may be taught or suggested herein. Further, though the systems and methods disclosed herein are described in connection with treatments of vertebrae, one of ordinary skill in the art will recognize that the systems and methods can also be used for the treatment of bone, generally, and are not limited for use in spinal treatment. In addition, while a number of variations of the invention have been shown and described in detail, other modifications and methods of use, which are within the scope of this invention, will be readily apparent to those of skill in the art based upon this disclosure. It is contemplated that various combinations or subcombinations of these specific features and aspects of embodiments may be made and still fall within the scope of the invention. Accordingly, it should be understood that various features and aspects of the disclosed embodiments can be combined with or substituted for one another in order to form varying modes of the discussed bone treatment systems and methods.

What is claimed is:

1. A medical apparatus for providing access to an interior portion of a bone, comprising:
an elongated sleeve configured for accessing a vertebral body, the sleeve defining a passageway extending from a proximal end to a distal open end of the sleeve, the sleeve having a first portion with a first cross-sectional area and a second portion with a second cross-sectional area, the second portion comprising threads about an exterior surface thereof and configured to engage bone, the second cross-sectional area being smaller than the first cross-sectional area; and
a seal element extending across the passageway, the seal configured to allow the insertion of an instrument through the passageway and distal open end of the sleeve.

2. The medical apparatus of claim 1, wherein the first portion has a mean cross-sectional diameter ranging from about 5 mm to 40 mm.

3. The medical apparatus of claim 2, wherein the first portion has a mean cross-sectional diameter ranging from about 8 mm to 25 mm.

4. The medical apparatus of claim 1, wherein the second portion has a mean cross-sectional diameter of less that about 8 mm.

5. The medical apparatus of claim 1, wherein the second portion has an axial length ranging between 2 mm and 15 mm.

6. The medical apparatus of claim 1, wherein the seal element is a flexible seal.

7. The medical apparatus of claim 1, wherein the seal element is an elastomeric seal.

8. The medical apparatus of claim 1, wherein the seal element is a hinged flap-type seal.

9. The medical apparatus of claim 1, wherein the seal element is disposed within the first portion.

10. The medical apparatus of claim 1, wherein the seal element is disposed at a proximal end of the first portion.

11. The medical apparatus of claim 1, wherein the bone-engaging threads have a self-tapping configuration.

12. The medical apparatus of claim 1, wherein the bone-engaging threads have a tapered cross-section.

13. The medical apparatus of claim 1, wherein the exterior surface of the tubular sleeve comprises an electrically insulative coating.

14. The medical apparatus of claim 1, wherein the sleeve passageway is defined by a surface comprising an electrically insulative coating.

15. The medical apparatus of claim 1, wherein at least a portion of the second portion comprises an electrically conductive material.

16. A system for providing access to an interior portion of a vertebra, comprising:
an elongated sleeve configured for accessing the interior of a vertebra, the sleeve having a handle end and a threaded distal end configured to threadably engage cortical bone of the vertebra, the sleeve defining a passageway extending from a proximal end to a distal open end thereof;, and
an elongated tool having a proximal end and sharp distal tip, the tool insertable through the passageway of the elongated sleeve and releasably lockable to the sleeve so that the distal tip extends outwardly of the distal open end of the sleeve by a selected dimension.

17. The system of claim 16, wherein the selected dimension is fixed.

18. The system of claim 16, wherein the selected dimension is less than about 30 mm.

19. The system of claim 16, wherein the selected dimension ranges from 1 mm to 15 mm.

20. The system of claim 16, wherein at least one of the handle end of the sleeve and the proximal end of the tool includes a locking mechanism.

21. The system of claim 20, wherein the locking mechanism comprises mating thread elements on the sleeve and tool.

22. The system of claim 16, further comprising means for releasably locking the elongated sleeve and the elongated tool relative to each other.

23. The system of claim 15 wherein the threaded distal end of the sleeve has a length ranging between 4 mm and 10 mm.

24. The system of claim 16, wherein the sleeve comprises a flexible seal disposed in the passageway configured to allow the tool to pass therethrough.

25. The system of claim 15, wherein the elongated sleeve is coupleable to an aspiration source for aspirating through the passageway of the sleeve.

26. A method for treating a vertebral body, comprising:
advancing an elongated sleeve through an incision in a patient's back to a vertebra;
engaging the elongated sleeve with a pedicle of the vertebra; and
inserting at least one tool through the elongated sleeve and into the vertebral body to perform at least one medical procedure.

27. The method of claim 26, wherein advancing includes penetrating through cortical bone of a pedicle of the vertebra with a sharp-tipped tool releasably coupled to the elongated sleeve.

28. The method of claim 27, wherein penetrating includes limiting the depth of penetration of the sharp-tipped tool to one of the posterior ⅔ of the vertebral body, the posterior ½ of the vertebral body, and the posterior ⅓ of the vertebral body.

29. The method of claim 26, wherein engaging comprises threadably engaging the sleeve to cortical bone of the vertebra.

30. The method of claim 26, further comprising injecting bone into the vertebral body.

31. The method of claim 26, wherein the medical procedure includes performing a biopsy.

32. A method of treating an abnormality in a bone, comprising:
providing a first flow of an exothermic polymer into the interior of a bone, the first flow exhibiting a first level of polymerization;
contemporaneously providing a second flow of a polymer into the bone, the second flow exhibiting a second level of polymerization, the first and second flows intermixing with each other; and
allowing complete polymerization of the first and second flows to thereby provide a polymer bone cement with enhanced functional properties.

33. The method of claim 32, wherein the enhanced physical property includes at least one of fatigue life, bending strength, compressive strength and tensile strength.

34. The method of claim 32, wherein providing the first flow comprises causing the first level of polymerization substantially by exothermic heating.

35. The method of claim 32, wherein providing the second flow comprises causing the second level of polymerization by applying thermal energy to the second flow.

36. The method of claim 35, wherein applying thermal energy to the second flow comprises applying at least one of: light energy, thermal energy from a resistive heating element, thermal energy from a heated vapor media, thermal energy from a radiofrequency source, thermal energy from a microwave source and thermal energy from an ultrasound source.

37. The method of claim 32 wherein applying thermal energy comprises applying energy from a resistive heating element comprising a PTCR material.

38. The method of claim 32, wherein the first flow comprises an interior portion of a flow of bone cement from an injector.

39. The method of claim 32, wherein the second flow comprises a surface portion of a flow of bone cement from an injector.
